(19) United States
(12) Patent Application Publication
(10) Pub. No.: US 2015/0265451 A1
(43) Pub. Date: Sep. 24, 2015

(54) Limb Immobilization Wrap

(71) Applicant: Paul Forster, Lutz, FL (US)

(72) Inventor: Paul Forster, Lutz, FL (US)

(21) Appl. No.: 14/659,700

(22) Filed: Mar. 17, 2015

Related U.S. Application Data
(60) Provisional application No. 61/954,657, filed on Mar. 18, 2014.

(51) Int. Cl. A61F 5/05

(52) U.S. Cl. CPC A61F 5/05; A61F 2210/0085

(57) ABSTRACT

A wrap for applying compression and for immobilizing a user's limb. The wrap comprises an elongated band of elastic material that is flexible so as to contour to the shape of a user's limb. The elongated band includes a plurality of semi-rigid strips arranged transversely thereon. The strips are preferably spaced from one another at a fixed interval and are parallel to one another. The elongated band includes an adhesive thereon for securing the wrap to a user's limb and for securing successive layers of the elongated band to one another when wrapped around a user's limb. In operation, the wrap can be wrapped in a spiral along the length of a user's limb in multiple layers in order to immobilize the limb, wherein the strips on successive layers are preferably oriented orthogonally to one another in order to provide rigidity to the wrap.

### CROSS REFERENCE TO RELATED APPLICATION

This application claims the benefit of U.S. Provisional Application No. 61/954,657 filed on Mar. 18, 2014. The above identified patent application is herein incorporated by reference in its entirety to provide continuity of disclosure.

### BACKGROUND OF THE INVENTION

1. Field of the Invention

The present invention relates to bandages and wraps. More specifically, the present invention provides an immobilizing wrap for maintaining a user's limb in a desired position. The wrap comprises an elongated band of material having a plurality of semi-rigid strips thereon for maintaining rigidity in the wrap. The wrap is adapted to be wrapped around a user's limb in order to immobilize the limb.

It is often necessary to immobilize a person's limb after a breakage, sprain, or other injury. Immobilizing the limb helps to facilitate healing of the person's limb by allowing the limb to rest. Further, immobilizing the limb helps to prevent the user from continuing to utilize the limb, which may worsen the injury or may result in further damage to the limb.

When a user's limb is injured, hard casts are often used to immobilize the user's limb. However, hard casts are generally suited for breakages and are not well suited for other injuries. Casts can be uncomfortable and do not provide cushioning for the user. Further, casts are not easily removed or reapplied, and prevent the user from being able to inspect the site of the injury. Alternatively, some people may use bandages to wrap a limb, however, bandages do not sufficiently immobilize a user's limb and do not provide sufficient support for the limb. Thus, a wrap for immobilizing a user's limb while providing comfort and convenience to the user is desired.

2. Description of the Prior Art

Devices have been disclosed in the prior art that relate to wound dressing and devices for immobilizing a limb of a user. These include devices that have been patented and published in patent application publications. These devices generally relate to wound dressing comprising elastic straps or bandages. The following is a list of devices deemed most relevant to the present disclosure, which are herein described for the purposes of highlighting and differentiating the unique aspects of the present invention, and further highlighting the drawbacks existing in the prior art.

One such device, U.S. Pat. No. 5,939,339 to Delmore et al. discloses an absorbent self-adhering elastic bandage. The bandage can be compressively wrapped around a wound and is adapted to absorb fluids. The bandage including a self-adhering elastic substance that does not adhere to clothing, hair, or skin. Thus, while Delmore et al. discloses an elastic bandage to be wrapped around a wound, Delmore et al. fails to disclose a plurality of transverse strips arranged on the bandage that add rigidity to the bandage.

Another device, U.S. Pat. No. 6,027,465 to Scholz et al. discloses a method of immobilizing a body member using a composite article. The article comprises a curable resin and a filler associated with the resin. The fillers add to the strength of the cured casting material. Moldable casting tapes are provided which comprise highly filled composite material. Thus, Scholz discloses a method for immobilizing a body member including curable resins and fillers, and does not disclose a bandage to be wrapped around a limb of a user.

U.S. Pat. No. 6,573,419 to Naimer discloses an elastic adhesive wound dressing for control of bleeding. The compression dressing comprises self-adhering elastic bandage strip designed to exert a compressive force when wrapped around a body part. Further, the dressing comprises an absorbent pad affixed to an inner side of the self-adhering elastic bandage strip. Thus, Naimer discloses a bandage for dressing wounds rather than for immobilizing a limb of a user, and Naimer fails to disclose the use of a plurality of semi-rigid strips on the bandage.

U.S. Pat. No. 6,464,658 to Darcey discloses a knee-immobilizer comprising an outer container composed of a moisture-resistant material and a plurality of elongated knee splint segments. Each knee splint includes an elongated substrate, a reactive system impregnated into the substrate. The system is stable when maintained in a moisture free condition and hardens when exposed to moisture. A flexible outer cover can be positioned over the substrate to enclose the substrate. Further, a bandage is provided for being positioned on the knee and adjacent the thigh and calf portions of the leg. Thus, Darcey discloses a knee immobilizer and fails to disclose an immobilizing wrap to be wrapped around any limb of a user and that can be secured thereto via an adhesive on the wrap.

U.S. Pat. No. 4,013,070 to Harroff discloses a body part immobilizer. The immobilizer comprises a wraparound immobilizer having interior and exterior stays that are adjustable to accommodate the size of the user. The stays may carry attachment straps by which the immobilizer is secured about the body part. Thus, Haroff discloses a wrap having straps for securement to a user, and does not disclose a wrap that can be wrap continually around a limb until the desired amount of compression or immobilizer is achieved.

Finally, U.S. Published Patent Application Number 2012/0101465 to McGuire, Jr. discloses a wound compression dressing. The wound compression dressing comprises an elastic layer, an adhesive layer on a first side of the elastic layer; and a release layer on the elastic layer. The dressing can be wrapped around a wound to provide direct compression thereto. McGuire, Jr., however, fails to disclose a dressing or bandage having a plurality of transverse strips arranged thereon for facilitating immobilization of a limb of a user.

These prior art devices have several known drawbacks. Devices in the prior art relate to wound dressings or bandages. The wound dressing are adapted to compress a wound and to absorb any fluids. These devices are not adapted to immobilize a user's limb. Devices that are adapted for immobilization fail to disclose a plurality of semi-rigid strips for immobilizing the limb of the user. Further, the devices do not allow the user to continually wrap a limb until the desired amount of immobilization or compression is achieved.

In light of the devices disclosed in the prior art, it is submitted that the present invention substantially diverges in design elements from the prior art and consequently it is clear that there is a need in the art for an improvement to existing immobilization wrap devices. In this regard the instant invention substantially fulfills these needs.

### SUMMARY OF THE INVENTION

In view of the foregoing disadvantages inherent in the known types of immobilization wraps now present in the prior art, the present invention provides a new immobilization wrap wherein the same can be utilized for providing convenience for the user when immobilizing a limb of a user and allowing the user to provide a desired amount of immobilization or compression.

It is therefore an object of the present invention to provide a new and improved immobilization wrap device that has all of the advantages of the prior art and none of the disadvantages.

It is another object of the present invention to provide an immobilization wrap device comprising an elongated band of elastic material having a plurality of semi-rigid strips thereon for providing rigidity, wherein additional rigidity can be applied by wrapping successive layers of the immobilization wrap around a limb in a spiral along the length thereof.

Another object of the present invention is to provide an immobilization wrap comprising an adhesive surface for easy securement to the user and to secure successive layers of the wrap.

Yet another object of the present invention is to provide an immobilization wrap that the user can wrap in multiple layers to provide a desired amount of compression or immobilization.

Another object of the present invention is to provide an immobilization wrap that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the outer surface of the immobilization wrap of the present invention.

FIG. 2 shows a cross sectional view of the immobilization wrap.

FIG. 3 shows a perspective view of the immobilization wrap as applied to a user's arm.

### DETAILED DESCRIPTION OF THE INVENTION

Reference is made herein to the attached drawings. Like reference numerals are used throughout the drawings to depict like or similar elements of the immobilization wrap of the present invention. For the purposes of presenting a brief and clear description of the present invention, the preferred embodiment will be discussed as used for immobilizing a limb of a user. The figures are intended for representative purposes only and should not be considered to be limiting in any respect.

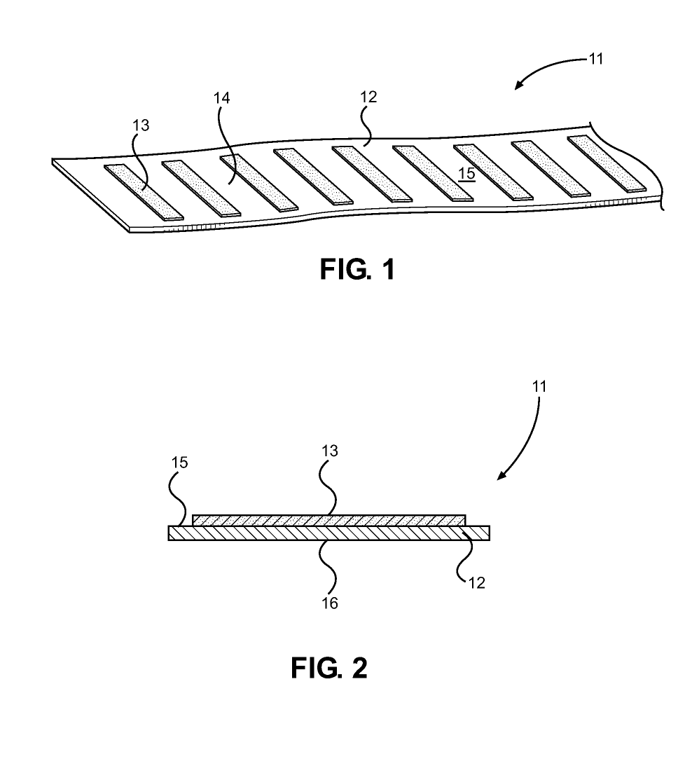

Referring now to FIG. 1, there is shown a perspective view of the outer surface of the immobilization wrap of the present invention. The immobilization wrap 11 comprises an elongated band 12 of an elastic material that is flexible so as to fit to the contours of a user's limb. The elongated band 12 can be used to apply compressive force to a user's limb for supporting and immobilizing the same. The elongated band 12 is preferably disposed in a roll for storage so that it can be easily dispensed and so that it is compact in configuration.

The elongated band 12 comprises an outer surface 15 having a plurality of semi-rigid strips 13 thereon. The strips 13 are arranged transversely to the longitudinal axis of the elongated band 12. The strips 13 are parallel to one another and are separated by a space 14 so that the elongated band 12 can contour to the limb being wrapped. Preferably, the strips 13 are spaced at a fixed interval. The strips 13 are of equal size, shape and dimension such that the strips 13 are substantially identical to one another.

Referring now to FIG. 2, there is shown a cross sectional view of the immobilization wrap. The wrap 11 comprises an elongated band 12 of elastic material that is flexible so as to contour to the user's limb. The outer surface 15 of the elongated band 12 comprises semi-rigid strips 13 permanently bonded thereon. The semi-rigid strips 13 are arranged transversely on the elongated band 12 and extend along the width thereof. The outer surface 15 and inner surface 16 of the immobilization wrap 11 include adhesive thereon for securement to the user's limb and for securement to additional layers of the elongated band 12. In this way, the wrap 11 can be securely positioned on a user and will remain in place during use.

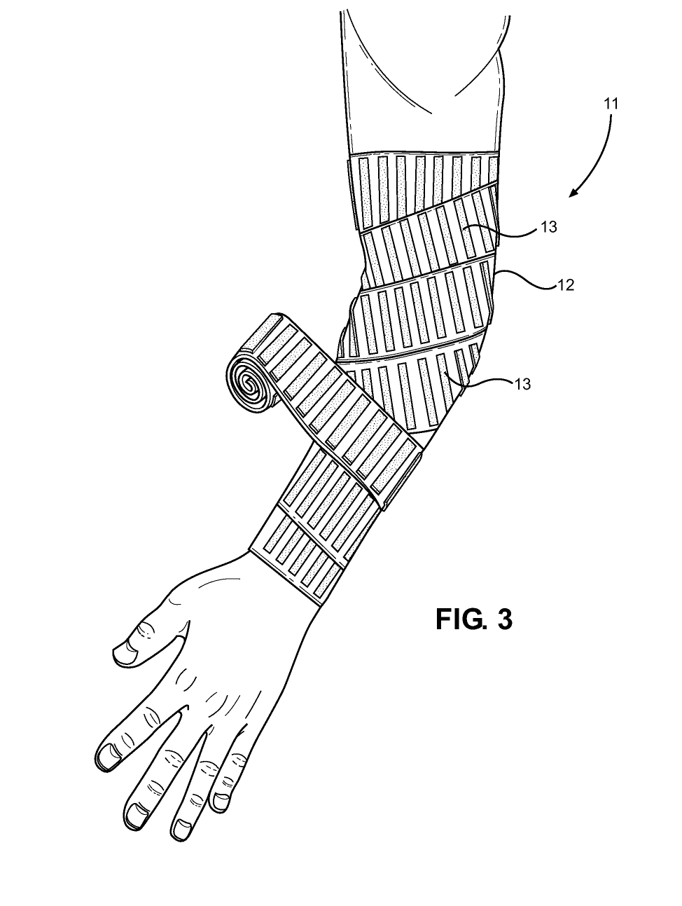

Referring now to FIG. 3, there is shown a perspective view of the immobilization wrap as applied to a user's arm. The immobilization wrap 11 can be wrapped around a limb of a user in order to immobilize the same. The elongated band 12 is flexible so that it can be continually wound around a limb of a user and arranged in the desired fashion. In operation, the user can spiral the immobilization wrap 11 around the limb along the length thereof so that the strips 13 thereon are oriented at an angle, preferably a forty-five degree angle, relative to the longitudinal axis of the limb. Once the limb has been wrapped around the length thereof, the user can spiral the immobilization wrap 11 around the limb in the opposite direction along the length thereof. In this way, the immobilization wrap 11 is applied in a first layer and a second layer wherein the strips 13 thereon cross one another such that they are substantially perpendicular to one another so as to create an immobilizing matrix. The user can wrap additional layers of the immobilization wrap 11 around the limb to increase the strength of the immobilizing matrix created by the overlapping strips 13.

The interior surface and exterior surface of the immobilization wrap comprises an adhesive thereon so that the layers of the wrap 11 will adhere to one another and so the wrap 11 will weakly adhere to the user's limb in order to maintain the wrap 11 in the desired position. The wrap 11 can be easily removed and reapplied as desired by the user. In this way, the user can inspect the injury and to replace the wrap with a new, clean wrap if the original wrap is damaged or soiled.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. An immobilization wrap for immobilizing a limb of a user, comprising:
an elongated band of elastic material having an inner surface and an outer surface;
a plurality of semi-rigid strips permanently affixed on said outer surface relative, wherein said plurality of semi-rigid strips are arranged transversely relative to a longitudinal axis of said elongated band;
an adhesive disposed on said inner surface and said outer surface of said elongated band.

2. The immobilization wrap of claim 1, wherein said elongated band of elastic material is adapted to apply compressive force to a user's limb when applied thereto.

3. The immobilization wrap of claim 1, wherein said plurality of semi-rigid strips are substantially equal in shape and dimension.

4. The immobilization wrap of claim 1, wherein said plurality of semi-rigid strips are spaced at a fixed interval from one another.

5. The immobilization wrap of claim 1, wherein said plurality of semi-rigid strips are parallel to one another.

6. The immobilization wrap of claim 1, wherein said elongated band is wound in a roll for storage.

7. A method of immobilizing a limb of a user, comprising:
using a wrap comprising an elongated band having a plurality of semi-rigid strips thereon;
wrapping said wrap around a user's limb in a plurality of layers;
securing said immobilization wrap on said user's limb via an adhesive on said immobilization wrap.

8. The method of immobilizing a limb of claim 7, wherein the step of wrapping said wrap around a user's limb in a plurality of layers includes the step of wrapping said wrap around a user's limb so that said plurality of semi-rigid strips are at a forty-five degree angle relative to the longitudinal axis of said user's limb.

9. The method of immobilizing a limb of claim 7, wherein the step of wrapping said wrap around a user's limb in a plurality of layers further includes the steps of:
wrapping said wrap around said user's limb in a first layer in a first direction, and
wrapping said wrap around said user's limb in a second layer in a second direction, such that said plurality of semi-rigid strips on said first layer and said plurality of semi-rigid strips on said second layer are perpendicular to one another.
